(19) United States
(12) Patent Application Publication
(10) Pub. No.: US 2018/0043088 A1
(43) Pub. Date: Feb. 15, 2018

(54) COMPACT MEDICAL INFUSION PUMPS

(71) Applicant: Smiths Medical ASD, Inc., Plymouth, MN (US)

(72) Inventors: Grant A. ADAMS, Anoka, MN (US); Eric J. WILKOWSKE, North Oaks, MN (US); Christopher A. LACY, Arden Hills, MN (US)

(73) Assignee: Smiths Medical ASD, Inc., Plymouth, MN (US)

(21) Appl. No.: 15/535,373

(22) PCT Filed: Dec. 2, 2015
(86) PCT No.: PCT/US15/63469

§ 371 Date: Jun. 12, 2017

Related U.S. Application Data
(60) Provisional application No. 62/094,519, filed on Dec. 19, 2014.

(51) Int. Cl. A61M 5/145; A61M 5/142

(52) U.S. Cl. CPC A61M 5/145; A61M 5/14216; A61M 2005/31518

(57) ABSTRACT

A compact medical infusion pump includes a base unit and a compact pump mechanism coupled to the base unit. The compact pump mechanism also includes a carriage member shaped to support a medical syringe, is movable in a first linear direction relative to the base unit, and is fixed to a first guide rod arm. The mechanism further includes a plunger driver shaped to selectively engage a plunger portion of the medical syringe, is moveable in a second linear direction opposite the first linear direction, and is fixed to a second guide rod arm. The mechanism also includes a rotatable drive member that is centrally located with respect to the base unit and is driven by a drive train assembly. The rotatable drive member engages both the first guide rod arm and the second guide rod arm to translate the carriage member and the plunger driver in opposite directions.

### TECHNICAL FIELD

Embodiments of this disclosure generally relate to compact medical infusion pumps. More particularly, embodiments of this disclosure relate to compact medical infusion pumps and related systems and methods, which can be used in or with syringe pumps, ambulatory infusion pumps, and similar medical infusion devices.

### BACKGROUND

In the field of medical infusion devices including “syringe pumps” and “ambulatory infusion pumps”, typically a pre-filled fluid syringe or reservoir is mechanically driven or controlled by a microprocessor to deliver a prescribed amount or dose of a drug or fluid at a controlled rate to a patient through an infusion line fluidly connected to the syringe or reservoir. Drugs or fluids delivered to a patient by way of syringe pumps and ambulatory infusion pumps can include, but are not limited to: therapeutic agents; nutrients; drugs; medicaments such as antibiotics, blood clotting agents, and analgesics; and other fluids. The devices can be used to introduce the drugs or fluids into patients' bodies utilizing any of several routes such as, for example, intravenously, subcutaneously, arterially, or epidurally.

Examples of syringe pumps and related components are disclosed in U.S. Pat. No. 4,978,335 titled “Infusion Pump with Bar Code Input to Computer,” U.S. Pat. No. 8,182,461 titled “Syringe Pump Rapid Occlusion Detection System,” and U.S. Pat. No. 8,209,060 titled “Updating Syringe Profiles for a Syringe Pump.” Each of these patents is hereby incorporated by reference in its entirety. As used throughout this disclosure, the term “syringe pump” is intended to generally pertain to any device which acts on a syringe to controllably force fluid outwardly therefrom. As used throughout this disclosure, the term “ambulatory infusion pump” is intended to generally pertain to any device that acts on a reservoir to controllably force fluid outwardly therefrom, or otherwise regulate a flow of fluid to an ambulatory patient.

As with other technologies, throughout the evolution of infusion devices there has been increasing demand for reduction in their physical dimensions and overall sizes. However, reducing the dimensions and sizes of infusion devices has been problematic. For example, syringe pump dimensions and sizes may be limited or dictated by syringe sizes and the size of components necessary to manipulate these syringes. A typical syringe pump has a lead screw that actuates a plunger driver mechanism, which in turn acts on a plunger in the syringe to move the plunger forwardly and thereby dispense fluid outwardly from the syringe. A relatively large syringe, such as, for example, a 60 mL syringe, can require 5 inches of linear movement or travel of the plunger driver to deliver an entire volume of fluid from the syringe. Thus, the pump would need to be sufficiently large to accommodate 5 inches of linear travel of the plunger. Furthermore, when a 60 mL syringe is full, it may have an effective length of about 10 inches resulting from a syringe column or reservoir length of 5 inches plus a corresponding plunger length of about 5 inches to provide travel forwardly within the reservoir to force fluid outwardly therefrom. Thus, when a full 60 mL syringe is installed in a syringe pump, a total linear distance occupied by the combination may exceed 10 inches. Not only can an extended syringe arrangement be problematic based on the considerable length of physical space occupied on one side of the pump, but further the stability and mechanical integrity of such an extended arrangement can also be problematic.

It would therefore be useful and advantageous to provide pump mechanisms for infusion devices, such as, for example, syringe pumps, which would be compact, convenient, and provide desired stability and mechanical integrity in accurately delivering infusates to patients.

### SUMMARY

This disclosure describes novel and inventive compact medical infusion pumps and related systems and methods, which can be used in or with syringe pumps, ambulatory infusion pumps, and similar medical infusion devices. In general, medical infusion pumps having split drive mechanisms provide compact pump arrangements beneficial to medical environments of limited space, and to stable, accurate fluid delivery.

In one embodiment, a compact medical infusion pump includes a base unit and a compact pump mechanism coupled to the base unit. The base unit includes a first stationary side panel and a second stationary side panel, and a drive train assembly generally centrally located in the base unit. The compact pump mechanism includes a carriage member, a plunger driver, and a rotatable drive member. The carriage member is shaped to support a medical syringe, movable in a first linear direction relative to the base unit, and fixed to a first guide rod arm that extends through the first stationary side panel. The plunger driver is shaped to selectively engage a plunger portion of the medical syringe, moveable in a second linear direction opposite the first linear direction, and fixed to a second guide rod arm that extends through the second stationary side panel. Further, the rotatable drive member is centrally located with respect to the base unit and driven by the drive train assembly. The rotatable drive engages both the first guide rod arm and the second guide rod arm to translate the carriage member and the plunger driver in opposite directions simultaneously, or approximately so, when rotated.

In another embodiment, a compact medical syringe pump includes a base unit, a slideable carriage assembly, and a slideable plunger assembly. The slideable carriage assembly supports and selectively translates a barrel portion of a syringe relative to the base unit. The slideable plunger assembly supports and selectively translates a plunger driver member that engages a plunger portion of the syringe. Further, the slideable carriage assembly moves in an oppositely-disposed, coordinated linear manner relative to the slideable plunger assembly, so as to control dispensing of fluid from the syringe. The slideable plunger assembly moves at an equal distance and speed to the slideable carriage assembly when expanded and retracted.

A further embodiment relates to a compact medical syringe pump, including a lower stationary base unit and an upper syringe manipulation assembly. The lower stationary base unit having a first side panel and a second side panel and a drive train assembly. The upper syringe manipulation assembly disposed above the lower stationary base unit in a two-part split structure that extends and retracts in accordance with the size of a syringe supported on the assembly. The upper syringe manipulation assembly is operatively coupled in an arrangement that extends and retracts equally from the first side panel and the second side panel of the stationary housing when adjusted.

An embodiment includes a compact medical syringe pump including a base unit and a compact pump mechanism. The compact pump mechanism is coupled to the base unit and includes a first longitudinal half screw, a second longitudinal half screw, a drive nut, a plunger driver, and a carriage. The first longitudinal half screw having a first thread orientation. The second longitudinal half screw having a second thread orientation that is opposite to the first thread orientation. The first and second half screws are substantially parallel to each other and together comprise a lead screw. The drive nut has an interior surface including both the first thread orientation and the second thread orientation, the nut being rotatably engaged with the first and second half screws. The carriage is coupled to the first half screw and the plunger driver is coupled to the second half screw. Further, when the drive nut rotates, the first half screw moves in a substantially linear direction and the second half screw simultaneously, or approximately so, moves in a substantially linear direction that is opposite to movement of the first half screw, with the carriage and the plunger driver thereby moving in substantially parallel, opposite directions corresponding to movements of the first and second half screws respectively.

In an embodiment, a compact pump mechanism includes a rotatable drive member, a first track, a second track, a plunger driver, and a carriage. The first track being movably engaged with the rotatable drive member, the first track further being longitudinally moveable by rotation of the rotatable drive member. The second track being movably engaged with the rotatable drive member, the second track further being substantially parallel to the first track and longitudinally moveable by rotation of the rotatable drive member. The carriage coupled to a first guide rod arm providing the first track. The plunger driver coupled to a second guide rod arm providing the second track. When the rotatable drive member rotates, the first track moves in a substantially linear direction and the second track simultaneously, or approximately so, moves in a substantially linear direction that is opposite to movement of the first track, with the carriage and the plunger driver thereby moving in substantially parallel, opposite directions corresponding to movements of the first and second tracks respectively.

Another embodiment includes a method of compact infusate delivery. The method includes loading a syringe having a barrel portion filled with fluid infusate and a plunger portion into a syringe pump having a split drive assembly. The method further includes moving a barrel portion of a syringe in a first direction relative to a base unit of a syringe pump using the split drive assembly. The method also includes moving a plunger portion of the syringe in a second direction, opposite that of the first direction, relative to the base unit of the syringe pump using the split drive assembly, the barrel portion and the syringe portion being moved in a simultaneous, or approximately so, coordinated fashion with respect to one another. The method also includes delivering the fluid infusate with the syringe pump.

### BRIEF DESCRIPTION OF THE DRAWINGS

The invention may be more completely understood in consideration of the following detailed description of various embodiments of the invention in connection with the accompanying drawings, in which:

FIG. 1 is an illustration of an example of a syringe pump of the prior art.

FIG. 2 is an example of a syringe pump including a compact pump mechanism, according to an embodiment.

FIG. 3 shows a cross-sectional perspective view of the syringe pump of FIG. 2 in which a top portion of the syringe pump has been removed, according to an embodiment.

FIG. 4 is a plan view of some components of an example of a compact pump mechanism, according to an embodiment.

FIG. 5 is a plan view of some components of an example of a compact pump mechanism, according to an embodiment.

FIG. 6 is an example of a syringe pump including a compact pump mechanism depicting some internal components of the compact pump mechanism within the syringe pump, according to an embodiment.

FIG. 7 is an example of a syringe pump including a compact pump mechanism, according to an embodiment.

FIG. 8 is an example of a syringe pump including a compact pump mechanism, according to an embodiment.

FIGS. 9A-C show an example of a cross-threaded nut arrangement for use in a compact pump mechanism, according to an embodiment.

### DETAILED DESCRIPTION

The various embodiments of the invention may be embodied in other specific forms without departing from the essential attributes thereof; therefore, the illustrated embodiments should be considered in all respects as illustrative and not restrictive.

Compact pump mechanisms described in greater detail by way of examples herein can be beneficial in numerous ways. For example, in various embodiments, a compact pump mechanism may reduce the overall size of a medical infusion pump, reduce a length of extension of a particular component from a pump housing in a particular direction, provide greater stability and mechanical integrity to components extending from a pump housing due to short cantilever length of support members, or provide a desirable centralized syringe and pump drive arrangement. As will be described by example herein, a compact pump mechanism can be achieved by effectively separating or “splitting” a medical infusion pump drive into two substantially parallel and oppositely-moving components. Accordingly, embodiments disclosed herein describing a “split drive” assembly, mechanism, or arrangement refer to embodiments in which an actuating member translates multiple non-continuous components to govern device motion.

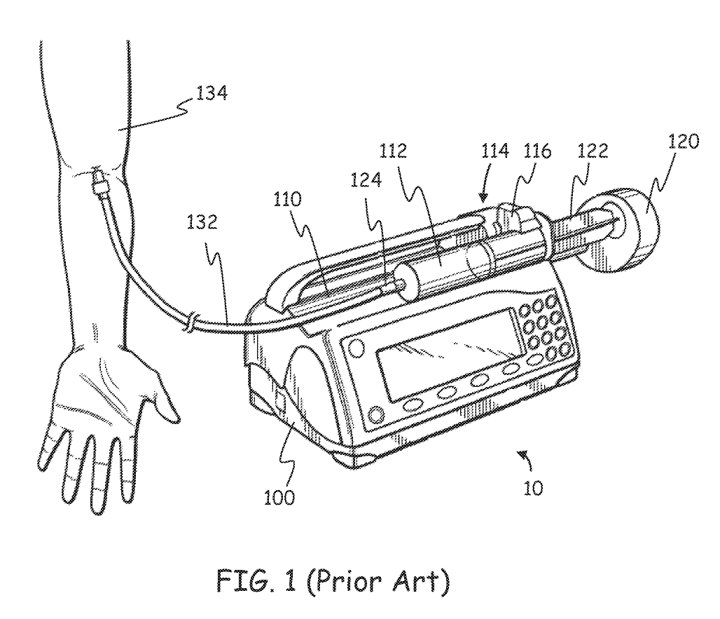

Referring to FIG. 1, an example of a syringe pump 10 of the prior art is shown. Typically, such a known pump 10 includes a base unit 100 having a user interface comprising a display screen and input controls such as push-buttons and the like as are visible in the drawing. Pump 10 also includes a curved surface or cradle 110 for receiving and supporting a barrel 112 of a syringe 114, a clamp 116 for selectively securing barrel 112 in cradle 110, and a plunger driver 120 for removably coupling a plunger 122 of syringe 114 to pump 10 and linearly driving plunger 122 within barrel 112. In use of pump 10, syringe 114 containing a desired volume of a flowable substance is installed by way of placement of barrel 112 in cradle 110, with barrel 112 being removably and selectively secured therein by clamp 116. Plunger driver 120 is removably coupled to a distal end of plunger 122 of syringe 114. Upon activation and operation of pump 10, driver 120 eventually advances forwardly (to the left in the drawing) which causes plunger 122 to also move forwardly in barrel 112 and thereby cause the flowable substance to be forced outwardly from syringe 114 at outlet 124. Tubing 132 is connected at outlet 124 to serve as a conduit for the flowable substance from syringe 114 to a patient 134.

In such known syringe pumps, a length of linear travel of plunger 122 in barrel 112 largely depends upon a corresponding possible length of linear travel of plunger driver 120 and that the entire length of travel of plunger driver 120 occurs in one direction. Thus, the overall dimensions of known syringe pumps are typically dependent upon maximum lengths of possible travel and directions of travel of their plunger drivers. With reference to FIG. 1, if plunger 122 of syringe 114 has a maximum travel of 5 inches within barrel 112, plunger driver 120 would therefore extend approximately 5 inches outwardly away from the pump (to the right in the drawing) when syringe 114 is installed in pump 10 as shown.

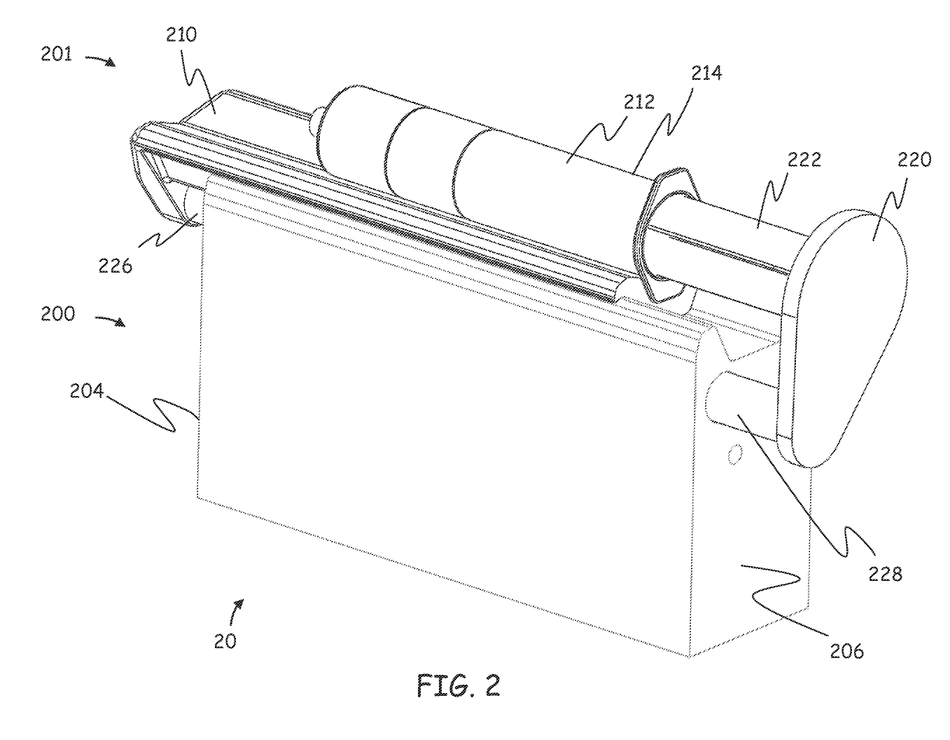

Referring now to FIG. 2, an example of a syringe pump 20 having a compact pump mechanism 201 is shown. The syringe pump 20 generally includes a base unit 200 (also alternatively referred to as a lower housing or a lower stationary base unit in this disclosure) and a compact pump mechanism 201 (also alternatively referred to as an upper syringe manipulation assembly in this disclosure). The base unit 200 is coupled to the compact pump mechanism 201, where the compact pump mechanism 201 is generally located above or partially within the upper portion of the base unit 200. Such a base unit 200 would typically be equipped with a user interface (not shown in FIGS. 2-3 and 6) comprising a display screen and input controls. The base unit 200 generally comprises a housing having a first stationary side panel 204 and a second stationary side panel 206 at opposite ends of the base unit 200. In the example of FIG. 2, the compact pump mechanism 201 includes a carriage 210 that supports a barrel 212 of a syringe 214, and a plunger driver 220 that removably couples a plunger 222 of the syringe 214 to pump 20. The syringe 214 is generally a replaceable component that removably fits within the compact pump mechanism 201 and is not necessarily or explicitly a component of the mechanism itself. In some embodiments, however, the syringe 214 may be considered part of the compact pump mechanism 201. The carriage 210 is at least partially supported by a first guide rod arm 226 that is generally parallel to the carriage 210 and extends through the first stationary side panel 204 of the base unit 200. The carriage 210 generally moves in a linear path in accordance with the positioning of first guide rod arm 226. The plunger driver 220 is supported by a second guide rod arm 228 that is generally disposed parallel to the first guide rod arm 226 and extends through the second stationary side panel 206 of the base unit 200. The plunger driver 220 generally moves in a linear path in accordance with the positioning of a second guide rod arm 228, with the path of linear travel of the carriage 210 generally being opposite that of the plunger driver 220. The path may be generally perpendicular to the disposition of the first and second stationary side panels 204 and 206 of the base unit 200 in some embodiments. Although not illustrated, a clamp could also be provided for removably securing the barrel 212 of the syringe 214 in carriage 210.

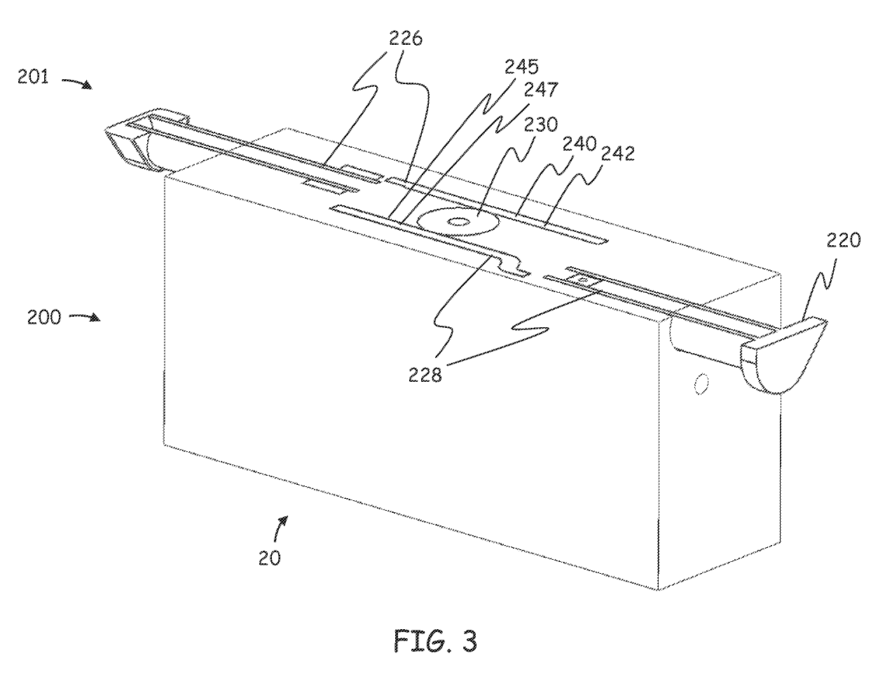

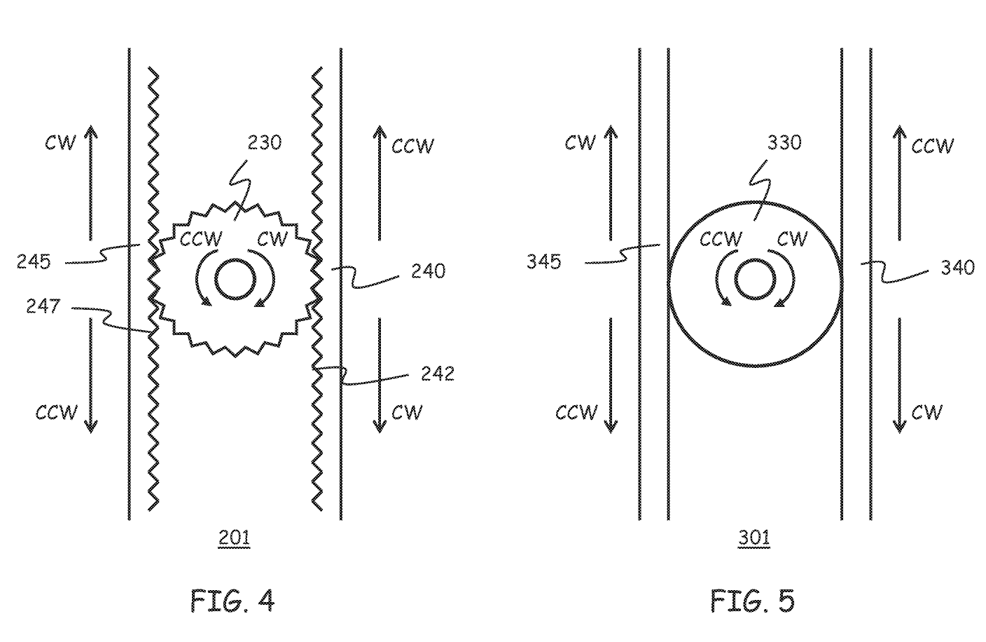

Generally internal to the compact pump mechanism 201 is a rotatable drive member 230, as shown in FIG. 3. Rotatable drive member 230 may be embodied in various shapes, designs, and configurations. In some embodiments, the rotatable drive member 230 may comprise a toothed sprocket as part of a rack and pinion type arrangement that includes the first guide rod arm 226 and second guide rod arm 228 although other shapes, designs, and configurations are possible as well. Mechanism 201 further includes a first track 240 as part of a first guide rod arm 226 that is movably engaged with rotatable drive member 230. As particularly depicted in FIG. 4, track 240 may include slots 242 that mechanically engage the toothed sprocket of rotatable drive member 230. Track 240 is thereby longitudinally moveable by rotation of rotatable drive member 230. Referring again to FIGS. 2 and 3, carriage 210 is coupled to first guide rod arm 226 and first track 240. Mechanism 201 further includes a second track 245 of second guide rod arm 228 that is also movably engaged with rotatable drive member 230. Similarly to first track 240, second track 245 includes slots 247 (again, as particularly depicted in FIG. 4) that mechanically engage the toothed sprocket of rotatable drive member 230. Second track 245 and second guide rod arm 228 are thereby also longitudinally moveable by rotation of rotatable drive member 230. Plunger driver 220 is coupled to track 245 of second guide rod arm 228.

With reference to FIGS. 2-4, it is to be appreciated and understood therefore that when rotatable drive member 230 rotates, first track 240 moves in a substantially linear direction and second track 245 simultaneously, or approximately so, moves in a substantially linear direction that is opposite to movement of first track 240. Thus carriage 210 and plunger driver 220, since they are coupled to tracks 240 and 245 respectively as aforesaid, move in substantially parallel but opposite directions corresponding to such opposite movements of their tracks 240 and 245 and guide rod arms 226 and 228, respectively.

When using syringe pump 20 and compact pump mechanism 201, a syringe 214 containing a desired volume of a flowable substance can be installed by way of removable placement or coupling of the syringe barrel 212 in carriage 210 (with, optionally, the barrel being secured by a clamp as aforementioned). Further, an end of a plunger 222 in the syringe 214 is removably coupled to plunger driver 220. After activation and during operation of pump 20, drive member 230 rotates which thereby causes tracks 240 and 245 to move in opposite directions. For example, when drive member 230 rotates in a clockwise (CW) direction as shown in the drawings, track 245 moves forwardly (to the left in FIGS. 2 and 3 or upwardly in FIG. 4) while track 240 moves backwardly (to the right in FIGS. 2 and 3 or downwardly in FIG. 4). Such movements would therefore advance plunger driver 220 forwardly (to the left in FIGS. 2 and 3) toward carriage 210, and simultaneously, or approximately so, move carriage 210 backwardly (to the right in FIGS. 2 and 3) toward plunger driver 220. Together, these movements would move the plunger forwardly in the barrel of the syringe and thereby cause the flowable substance to be forced outwardly therefrom as desired. It is to be appreciated and understood, therefore, that a compact pump mechanism, as described by example or otherwise contemplated herein, effectively provides a medical infusion pump that enables a full range of plunger travel in a device that can be relatively smaller than known pumps.

It is also to be appreciated and understood that mechanism 201 can be used for reversing direction of a plunger's travel such as when, for example, an occlusion is detected by the pump and the plunger is commanded to, intentionally, move backwardly or retreat a desired distance until the occlusion has been removed. In such an occurrence, drive member could be commanded to rotate in a counter-clockwise (CCW) direction as shown in FIGS. 2-4, which would cause track 245 to move backwardly (to the right in FIGS. 2 and 3 or downwardly in FIG. 4) while track 240 moves forwardly (to the left in FIGS. 2 and 3 or upwardly in FIG. 4). Such movements would therefore move plunger driver 220 backwardly (to the right in FIGS. 2 and 3) away from carriage 210, and simultaneously, or approximately so, move carriage 210 forwardly (to the left in FIGS. 2 and 3) away from plunger driver 220. Together, these movements would move the plunger backwardly in the barrel of the syringe and thereby stop, or possibly even reverse, the flow of the flowable substance from the syringe as may be desired in a particular situation.

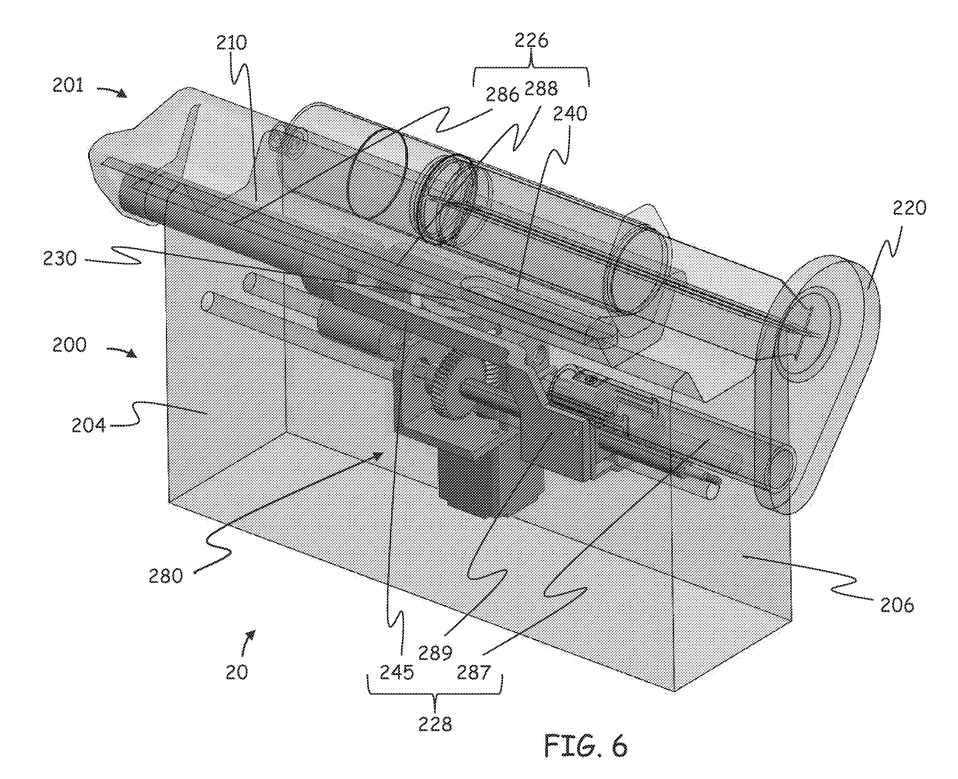

Referring now to FIG. 5, therein illustrated is another example of certain components of a compact pump mechanism 301. In this example of mechanism 301, although not specifically illustrated but similarly to FIGS. 2 and 3, a carriage supports a barrel of a syringe and a plunger driver removably couples a plunger of the syringe to a pump including mechanism 301. A clamp could also be provided for removably securing the barrel of the syringe in the carriage. Mechanism 301 includes a rotatable drive member 330. In this example, rotatable drive member 330 comprises a magnetic component. Mechanism 301 further includes a first track 340 that is movably engaged with rotatable drive member 330. First track 240 includes a material that magnetically engages the magnetic component of rotatable drive member 330. Track 340 is thereby longitudinally moveable by rotation of drive member 330, with the carriage (not illustrated) coupled to first track 340. Mechanism 301 further includes a second track 345 that is also movably engaged with rotatable drive member 330. Similarly to first track 340, second track 345 includes a material that magnetically engages the magnetic component of rotatable drive member 330, with the plunger driver (not illustrated) coupled to second track 345. When rotatable drive member 330 rotates, first track 340 moves in a substantially linear direction and second track 345 simultaneously, or approximately so, moves in a substantially linear direction that is opposite to movement of first track 340. Thus the carriage and the plunger driver, since they are coupled to tracks 340 and 345 respectively as aforesaid, move in substantially parallel but opposite directions corresponding to such opposite movements of their tracks 340 and 345 and guide rod arms 226 and 228. Use of a pump with compact pump mechanism 301 for a syringe containing a flowable substance would be analogous to pump 20 with mechanism 201 as aforedescribed. Mechanism 301 can be used for reversing direction of a plunger's travel, analogously to pump 20 with mechanism 201 as aforedescribed.

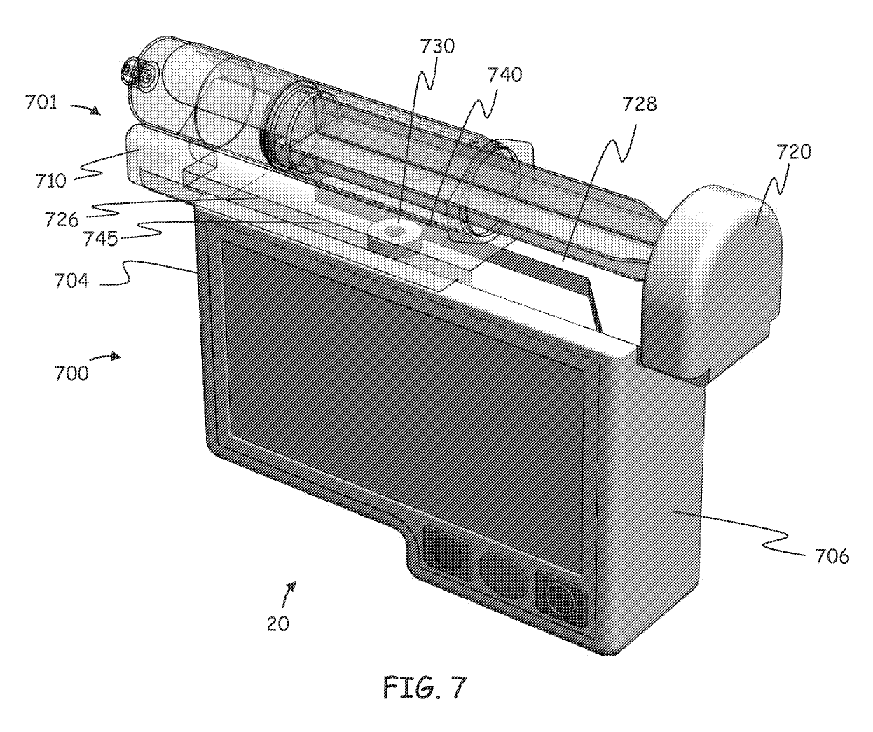

FIGS. 6-8 show other examples of medical infusion pumps with compact pump designs. FIG. 6 shows an internal view of the syringe pump 20 in which the drive train assembly 280 can be seen. The drive train 280 assembly is generally centrally located in the base unit 200 between the stationary side panels 204 and 206. The drive train 280 comprises the motor, gears, and other components needed to drive the rotatable drive member 230, including guide rod arms 226 and 228 and associated tracks 240 and 245. For purposes of this disclosure the first guide rod arm 226 includes a rod-like portion 286 which extends internally and externally through the stationary side panel 204 of the base unit 200. In this example, the first guide rod arm 226 further includes a multifaceted arm structure 288 that connects with the rod-like portion 286. First guide rod arm 226 also includes first track 240 that interfaces with the rotatable drive member 230. Accordingly, the combination of the first track 240, rod like portion 286, and multifaceted arm structure 288 comprises a first guide rod arm 226. Similarly, the combination of second track 245, rod-like portion 287, and multifaceted arm structure 289 comprise the second guide rod arm 228. Guide rod arms 226 and 228 can be embodied in various shapes and sizes in various embodiments and are not limited to those structures disclosed herein.

The central location of the drive train 280 and centralized drive movement of the rotatable drive member 230 provides a number of advantages. Known syringe pumps and similar devices generally position the motor and drive at one side of a pump housing unit in order to have a sufficiently long distance of possible plunger driver travel in one direction from a stationary or otherwise fixed carriage relative to base unit 200 to accommodate a fully extended or un-advanced syringe plunger with, for example, a filled syringe that is ready for use in dispensing a medicament contained in the syringe to a patient. Past guide rod arm members would extend a considerable distance from the drive component of the motor that was roughly equivalent to the length of such an extended or un-advanced syringe plunger.

Those of skill in the infusion arts will also appreciate that, although somewhat supported internally or structurally, typical cantilever arm lengths are significant in extension of plunger drivers in known pumps. Long cantilever arms supporting the plunger drivers of known pumps have potentially caused operational disadvantages related to, for example, stability and precision of components in those pumps. But in comparison, the presently disclosed examples of a split-drive arrangement advantageously includes two relatively short guide rod arms 226 and 228. Each of these guide rod arms 226 and 228 provide, as compared to known pumps, a much reduced cantilever arm extending from the central rotatable drive member 230 or respective stationary side panel 206 at one side to the plunger driver 220. Likewise the cantilever arm extending from the central rotatable drive member 230 or side panel 204 at one side to the end portion of the carriage 210 provides a much reduced length as comparted to known pumps. Accordingly, greater stability and accuracy can be achieved when a mechanism with reduced cantilever arms extend from the base unit 200. In some embodiments, the length of the second guide rod arm 228 extending between stationary side panel 206 of the base unit 200 and the plunger driver 220 serves as a cantilever arm having a length less than the length of the plunger portion 222 of the medical syringe 214.

Moreover, it is to be appreciated and understood that the novel and inventive arrangement of components according to subject matter hereof generally provides for approximately equal but opposite linear travel of the plunger driver 220 and carriage member 210 in a coordinated fashion from either side of the base unit 200 depending upon the size of the inserted syringe. In some embodiments, the first guide rod arm 226 extends partially beyond the first stationary side panel 204 and the second guide rod arm 228 extends partially beyond the second stationary side panel 206 when the syringe 14 is full and the plunger 222 extends outwardly from barrel 212. The disclosed arrangement does not largely extend only one portion of the pump mechanism 201 from only one side of the pump 20. Accordingly, any potential interference caused by extending features would generally be balanced and more restricted to the immediate proximity of the base unit 200 of the pump 20 itself due to centering. The pump 20 is largely a self-centered device with respect to lateral displacement of components from the sides. As compared to mechanisms of known pumps, this centering effect provides convenient and compact syringe pumps that are less likely to interfere with other devices and medical professionals attending to a patient connected to the novel and inventive pumps described by example or otherwise contemplated herein. The compactness provided can be extremely important in environments, such as emergency room settings, in which numerous devices and medical professionals are surrounding a patient and thus physical space is limited.

Accordingly, in some embodiments the compact medical syringe pump 20 includes a lower stationary base unit 201 with side panels 204 and 206 on the sides of a drive train assembly 280 that is generally centered in the base unit 201 between these side panels. Located above the base unit 201 is an upper syringe manipulation assembly 201 (or compact pump mechanism) that includes a two-part split structure that extends and retracts in accordance with the size or contained medicament volume of a syringe 214 thereby supported. The upper syringe manipulation assembly 201 is operatively coupled to extend and retract equally from the first side panel 204 and the second side panel 206 of the base unit 200 when the assembly is adjusted. Moreover, the upper syringe manipulation assembly 201 provides two separate cantilever support arms to support a syringe coupled to the two-part split structure.

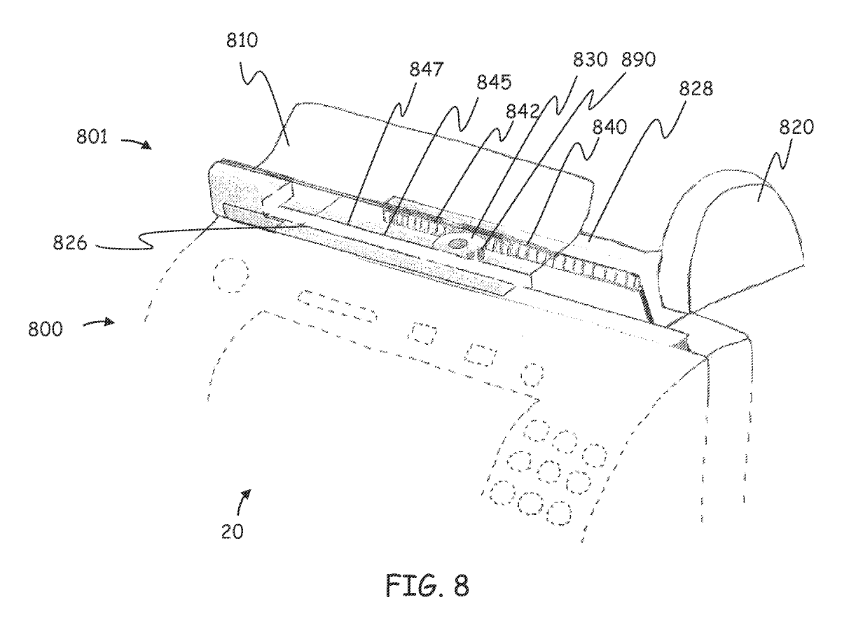

FIG. 7 shows another example of an embodiment of a compact medical syringe pump 20 having a base unit 700 and compact pump mechanism 701 generally similar to that disclosed in FIG. 2. The compact pump mechanism 701, however, contains a split drive with arm members 726 and 728 associated with the opposite sides of rotatable drive member 730. For example, the first drive arm 726 that supports the carriage 710 includes and is associated with a track 745 located on the near side of the device in the drawing. The second drive arm 728 that supports the plunger driver 720 includes and is associated with the track 740 located on the far side of the pump. In general, however, rotation of the rotatable drive member 730 effectively urges the carriage 710 and plunger driver 720 either toward one another or away from one another depending upon the direction of rotation. Another feature that can be seen in the pump 20 of FIG. 7 is a compact pump mechanism 701 that is able to retract the plunger driver 720 and end of the carriage 710 to a recessed arrangement. Specifically, they are recessed to be flush with or narrower than the ends of stationary side panels 704 and 706. In such an embodiment, not even the plunger driver 720 will cause protrusions or interference beyond the spatial footprint of the base unit 700.

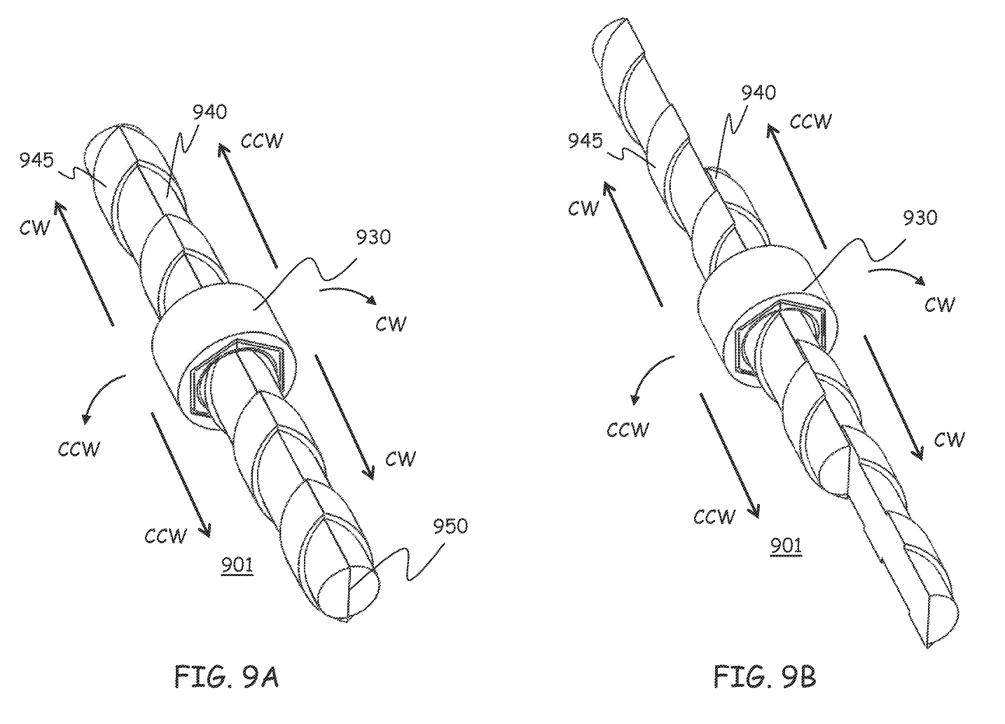

FIG. 8 illustrates another embodiment of a compact medical syringe pump 20 having a base unit 800 and compact pump mechanism 801 generally similar to that disclosed in FIG. 7. The compact pump mechanism 801, specifically depicts the tracks 840 and 845 on the guide rod arms 826 and 828 in greater detail. As shown on in FIG. 8, the slots 842 and 847 are able to mate with and interact with the teeth 890 of the rotatable drive member 830. Slots and corresponding teeth on the rotary drive member 830 can be varied to best accommodate the type of precise motion required.

Although not illustrated in FIGS. 2-8, it is to be understood that the aforedescribed examples of tracks in compact pump mechanisms could be moveably coupled or slideably secured in pumps in which compact pump mechanisms have been installed by way of, for example, longitudinal channels or slots formed in surfaces of the pumps on opposite sides of the rotatable drive members. Furthermore, but although also not illustrated in FIGS. 2-8, it is to be understood that rotation of the rotatable drive members could be provided by, for example, electrically-powered stepper motors in the pumps that are electro-mechanically coupled to the rotatable drive members by any suitable techniques.

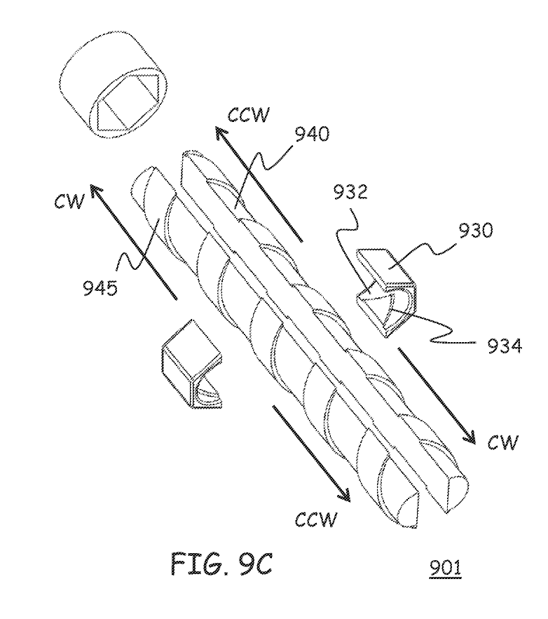

Referring now to FIGS. 9A-9C, therein illustrated is another example of a compact pump mechanism 901. Specifically, FIG. 9A discloses a central cross threaded nut mechanism in assembled relation. FIG. 9B discloses the central cross threaded nut mechanism in an assembled relation in which a portion of the mechanism has advanced axially in opposing forward and backward directions based upon rotation of the central nut. FIG. 9C discloses the central cross threaded nut mechanism in an exploded view, such that each of the components can be better understood. Such a cross threaded nut mechanism could be implemented within a syringe pump to replace the centralized moving structure of a compact pump mechanism. For example, instead of a centrally mounted rack and pinion type assembly as disclosed by example in FIGS. 1-8 herein, the cross thread nut mechanism could replace the rotatable drive mechanism with a nut that is driven by rotation proximate the center of the base unit. Further the guide rod arms could be at least partially replaced by the half lead screw structures discussed below. Using this cross threaded nut arrangement allows for another type of split drive device that can effectively provide a pump with enhanced compactness and advantageous shape.

In this example of mechanism 901, although not specifically illustrated but similar to FIGS. 2 and 3, a carriage would support a barrel of a syringe and a plunger driver would removably couple a plunger of the syringe to a pump including mechanism 901. Further, a clamp could also be provided for removably securing the barrel of the syringe in the carriage. The mechanism 901 shown in FIGS. 9A-C includes a drive nut 930 having an interior surface that includes a first thread orientation 932 and a second thread orientation 934 (as shown, in particular, in FIG. 9C). In this example, the first thread orientation comprises left-handed threads and the second thread orientation comprises right-handed threads. Mechanism 901 further includes a first longitudinal half screw 940 having a first thread orientation or left-handed threads, and a second longitudinal half screw 945 having a second thread orientation or right-handed threads. First and second half screws 940 and 945 are substantially parallel to each other and together comprise a lead screw 950 (as shown, in particular, in FIG. 9A). Drive nut 930, having an interior surface that includes both the first and second thread orientations or left-handed and right-handed threads as aforesaid, is thereby rotatably engaged with first and second half screws 940 and 945 having corresponding left-handed and right-handed threads as aforesaid, respectively. Although not illustrated, it is to be understood that the plunger driver and any associated guide rod arm can be coupled to first half screw 940, and the carriage and any associated guide rod arm can be coupled to second half screw 945. When drive nut 930 rotates, half screw 940 moves in a substantially linear direction and half screw 945 simultaneously, or approximately so, moves in a substantially linear direction that is opposite to movement of half screw 940. Thus the plunger driver and carriage, since they are coupled to half screws 940 and 945 respectively as aforesaid, move in substantially parallel but opposite directions corresponding to such opposite movements of their half screws 940 and 945. Use of a pump with compact pump mechanism 901 for a syringe containing a flowable substance would be analogous to pump 20 with mechanisms 201 or 301 as aforedescribed; and mechanism 901 can be used for reversing direction of a plunger's travel, analogously to pump 20 with mechanisms 201 and 301 also as aforedescribed.

Although not illustrated in FIGS. 9A-9C, it is to be understood that rotation of the drive nuts could be provided by, for example, electrically-powered stepper motors in the pumps that are electro-mechanically coupled to the drive nuts by any suitable techniques.

Accordingly, operation of compact infusate delivery of many of the above embodiments of a compact syringe can be carried out by an operator accordingly to a number of steps. First, a syringe having a barrel portion filled with fluid infusate and a plunger portion is loaded into a syringe pump having a split drive assembly. A barrel portion of a syringe is moved in a first direction relative to a base unit of a syringe pump using the split drive assembly. A plunger portion of the syringe is moved in a second direction, opposite that of the first direction, relative to the base unit of the syringe pump using the split drive assembly. This is done such that the barrel portion and the plunger portion are moved in a simultaneous, or approximately so, coordinated fashion with respect to one another. The fluid infusate accordingly is able to be delivered by the syringe pump.

Irrespective of a particular embodiment, it is to be appreciated and understood that compact pump mechanisms that have been described by example, or which are otherwise contemplated herein, can be characterized in that they provide movement of syringe barrels and plungers at substantially equal rates, but in linearly opposite directions. Thus, these novel and inventive compact pump mechanisms thereby provide substantially steady-state rates of delivery of flowable substances outwardly from the syringes.

It is also to be appreciated and understood that types, components, dimensions, fabrication processes, and other particulars and parameters of aforedescribed example embodiments can be substituted for others as desired, or that accessories can be added thereto. For example, the tracks could have any desired lengths provided that they are compatible with length dimensions of pumps in which they are installed.

While compact pump mechanisms have been particularly shown and described with reference to the accompanying figures and specification, it should be understood however that other modifications thereto are of course possible; and all of them are intended to be within the true spirit and scope of novel and inventive devices described herein. Thus, configurations and designs of various features could be modified or altered depending upon particular embodiments. For example, the carriage and the plunger driver could be coupled to the tracks in any order. Thus, although some examples herein have described the first tracks as being coupled to carriages and the second tracks as being coupled to plunger drivers, the first tracks could instead be coupled to plunger drivers with the second tracks therefore coupled instead to carriages. In such embodiments, the CW and CCW movements of the rotatable drive members would result in movements of the tracks, and their coupled carriages and plunger drivers, that would be analogous but opposite to the aforedescribed examples.

Compact pump mechanisms as described by example or otherwise contemplated herein could also include combinations of the aforedescribed examples of rotatable drive members having toothed sprockets or magnetic components, and tracks having slots or materials that magnetically engage the magnetic components, respectively. In those embodiments, magnetic sprockets could be coupled to slotted tracks having materials that magnetically engage the magnetic sprockets, with such compact pump mechanisms possibly being less susceptible to vibration and external forces than, for example, conventional pump mechanisms.

Furthermore, although not illustrated, compact pump mechanisms as described by example or otherwise contemplated herein could also include suitable vernier or “fine adjustment” controls for or with the rotatable drive members, tracks, drive nuts, and half screws, to possibly enable more precise movement of these components when in use.

Regardless of particular components or modes of action, it is to be appreciated and understood that compact pump mechanisms—such as have been described by example or are otherwise contemplated herein—can provide pump mechanisms for infusion devices which would be relatively compact and which would not be necessarily be defined in dimensions or sizes by syringes installed therein.

It is also to be appreciated and understood that compact pump mechanisms as have been described by example or otherwise contemplated herein could potentially be used for or with virtually any devices which control the delivery or movement of flowable substances from one location to another.

It is further to be understood that dimensioning and scaling of the drawings herein have been chosen to clearly show details of example embodiments. Thus, in some embodiments it is possible that spacing between, or orientations of, various features might be variable and visually different from those illustrated. In any event, dimensioning and scaling could vary significantly across various embodiments of compact pump mechanisms.

It is additionally to be understood in general that any suitable alternatives may be employed to provide novel and inventive compact pump mechanisms such as those that are described by example or otherwise contemplated herein.

Lastly, compositions, sizes, and strengths of various aforementioned components of compact pump mechanisms that are described by example or otherwise contemplated herein are all a matter of design choice depending upon intended uses thereof.

Accordingly, these and other various changes or modifications in form and detail may also be made, without departing from the true spirit and scope of compact pump mechanisms that may be defined by the appended claims.

It should also be appreciated that the exemplary embodiment or exemplary embodiments are only examples, and are not intended to limit the scope, applicability, or configuration of the invention in any way. Rather, the foregoing detailed description will provide those skilled in the art with an enabling disclosure for implementing the exemplary embodiment or exemplary embodiments. It should be understood that various changes can be made in the function and arrangement of elements without departing from the scope of the invention as set forth in the appended claims and the legal equivalents thereof. For example, in embodiments described with a syringe-type infusion pump, it is to be understood that an ambulatory type pump could have been alternatively employed.

The embodiments above are intended to be illustrative and not limiting. Additional embodiments are within the claims. Although the present invention has been described with reference to particular embodiments, workers skilled in the art will recognize that changes may be made in form and detail without departing from the spirit and scope of the invention.

Various modifications to the invention may be apparent to one of skill in the art upon reading this disclosure. For example, persons of ordinary skill in the relevant art will recognize that the various features described for the different embodiments of the invention can be suitably combined, un-combined, and re-combined with other features, alone, or in different combinations, within the spirit of the invention. Likewise, the various features described above should all be regarded as example embodiments, rather than limitations to the scope or spirit of the invention. Therefore, the above is not contemplated to limit the scope of the present invention.

1. A compact medical infusion pump, comprising:
a base unit including a first stationary side panel and a second stationary side panel, and a drive train assembly generally centrally located in the base unit; and
a compact pump mechanism coupled to the base unit, including:
a carriage member shaped to support a medical syringe, movable in a first linear direction relative to the base unit, and fixed to a first guide rod arm that extends through the first stationary side panel;
a plunger driver shaped to selectively engage a plunger portion of the medical syringe, moveable in a second linear direction opposite the first linear direction, and fixed to a second guide rod arm that extends through the second stationary side panel; and
a rotatable drive member centrally located with respect to the base unit and driven by the drive train assembly, that engages both the first guide rod arm and the second guide rod arm to translate the carriage member and the plunger driver in opposite directions when rotated.

2. The compact medical infusion pump of claim 1, wherein the second guide rod arm has a length extending between a stationary side panel of the base unit and the plunger driver serves as a cantilever arm having a length less than the length of the plunger portion of the medical syringe.

3. The compact medical infusion pump of claim 1, wherein the first guide rod arm extends partially beyond the first stationary side panel and the second guide rod arm extends partially beyond the second stationary side panel when the medical syringe is full.

4. The compact medical infusion pump of claim 1, wherein the rotatable drive member provides a rack and pinion arrangement with the first guide rod arm and the second guide rod arm.

5. A compact medical infusion pump, comprising:
a base unit;
a slideable carriage assembly that supports and selectively translates a barrel portion of a syringe relative to the base unit;
a slideable plunger assembly that supports and selectively translates a plunger driver member relative to the base unit that engages a plunger portion of the syringe,
wherein the slideable carriage assembly moves in an oppositely-disposed, coordinated linear manner relative to the slideable plunger assembly, so as to control dispensing of fluid from the syringe.

6. The compact medical infusion pump of claim 5, wherein the slideable plunger assembly moves at an equal distance and speed to the slideable carriage assembly when expanded or retracted.

7. The compact medical infusion pump of claim 5, wherein the slideable carriage assembly is coupled to the slideable plunger assembly based on a rack and pinion arrangement.

8. A compact medical infusion pump, comprising:
a lower stationary base unit having a first side panel and a second side panel and a drive train assembly;
an upper syringe manipulation assembly disposed above the lower stationary base unit in a two-part split structure that extends and retracts in accordance with the size of a syringe supported on the upper syringe manipulation assembly;
wherein the upper syringe manipulation assembly is operatively coupled in an arrangement that extends and retracts equally from the first side panel and the second side panel of the lower stationary housing when adjusted.

9. The compact medical infusion pump of claim 8, wherein the upper syringe manipulation assembly provides two separate cantilever arms to support the syringe along its length.

10. A compact medical infusion pump, comprising:
a base unit;
a compact pump mechanism coupled to the base unit, including:
a first longitudinal half screw having a first thread orientation, and a second longitudinal half screw having a second thread orientation that is opposite to the first thread orientation, the first and second longitudinal half screws being substantially parallel to each other and together comprising a lead screw;
a drive nut having an interior surface including both the first thread orientation and the second thread orientation, the drive nut being rotatably engaged with the first and second longitudinal half screws;
a carriage coupled to the first longitudinal half screw; and
a plunger driver coupled to the second longitudinal half screw,
wherein when the drive nut rotates, the first longitudinal half screw moves in a substantially linear direction and the second longitudinal half screw moves in a substantially linear direction that is opposite to movement of the first longitudinal half screw, with the carriage and the plunger driver thereby moving in substantially parallel, opposite directions corresponding to movements of the first and second longitudinal half screws respectively.

11. The compact medical infusion pump of claim 10 wherein the carriage is sized to support a syringe comprised of a plunger and a barrel containing a flowable substance, with the plunger being removably coupled to the plunger driver and the barrel being removably coupled to the carriage, wherein when the drive nut rotates, the plunger moves within the barrel of the syringe.

12. The compact medical infusion pump of claim 11, wherein the barrel and the plunger of the syringe move at substantially equal rates but in linearly opposite directions, thereby providing a substantially steady-state rate of delivery of the flowable substance outwardly from the syringe.

13. A compact pump mechanism, comprising:
a rotatable drive member;
a first track movably engaged with the rotatable drive member, the first track being longitudinally moveable by rotation of the rotatable drive member;
a second track movably engaged with the rotatable drive member, the second track being substantially parallel to the first track and longitudinally moveable by rotation of the rotatable drive member;
a carriage coupled to a first guide rod arm providing the first track; and
a plunger driver coupled to a second guide rod arm providing the second track,
wherein when the rotatable drive member rotates, the first track moves in a substantially linear direction and the second track moves in a substantially linear direction that is opposite to movement of the first track, with the carriage and the plunger driver thereby moving in substantially parallel, opposite directions corresponding to movements of the first track and the second track respectively.

14. The compact pump mechanism of claim 13, wherein:
the rotatable drive member comprises a sprocket;
the first track includes slots that mechanically engage the sprocket of the rotatable drive member; and
the second track includes slots that mechanically engage the sprocket of the rotatable drive member.

15. The compact pump mechanism of claim 13, wherein:
the rotatable drive member comprises a magnetic component;
the first track includes material that magnetically engages the magnetic component of the rotatable drive member; and
the second track includes material that magnetically engages the magnetic component of the rotatable drive member.

16.-20. (canceled)
